(19) United States
(12) Patent Application Publication
(10) Pub. No.: US 2024/0131297 A1
(43) Pub. Date: Apr. 25, 2024

(54) VALVE CASSETTE FOR A TURBINE MECHANICAL VENTILATOR METHOD FOR HOLDING AIRWAY PRESSURE

(71) Applicant: Flight Medical Innovations Ltd., Petach-Tikva (DE)

(72) Inventors: Amir KLEINSTERN, Kiryat-Ono (IL); Yossi HALFON, Givon HaHadasha (IL); Nir DORHAM, Ramat Gan (IL)

(73) Assignee: Flight Medical Innovations Ltd., Petach-Tikva (IL)

(21) Appl. No.: 18/277,586

(22) PCT Filed: Feb. 18, 2021
(86) PCT No.: PCT/IL2021/050191

§ 371 Date: Aug. 16, 2023

(51) Int. Cl. A61M 16/20; A61M 16/00; F16K 7/12; F16K 15/03

(52) U.S. Cl. CPC A61M 16/208; A61M 16/0066; F16K 7/12; F16K 15/035; A61M 2016/0027; A61M 2016/003; A61M 2205/121; A61M 2205/128

(57) ABSTRACT

A valve cassette for a turbine mechanical ventilator includes a plurality of diaphragm check valves, a plate including a plurality of seats configured for receiving said plurality of diaphragm check valves, and at least one connecting element configured for fixing valve cassette to a turbine housing inlet. Each of said plurality of diaphragm check valves includes a flexible membrane and a plug. Each of said plurality of seats includes a bore for receiving said plug and a plurality of openings through which air is configured to flow through the valve cassette.

### FIELD AND BACKGROUND OF THE INVENTION

The present invention, in some embodiments thereof, relates to a turbine mechanical ventilator and, more particularly, but not exclusively, to a valve cassette for a turbine mechanical ventilator and method for holding airway pressure therewith.

Conventional ventilators are known to require compressed air as a drive gas as well as a high-pressure oxygen source to operate. In many areas, a central supply infrastructure is not available to support operation of such ventilators. Turbine mechanical ventilators, on the other hand, are electrically powered and are operated with ambient air. High or low pressured oxygen can be mixed with the ambient air drawn in by the turbine before it is directed to the patient. Such turbine mechanical ventilators are relatively lightweight, portable and can be quickly shifted and readily installed as needed.

The demand for turbine mechanical ventilators has risen dramatically over the course of the COVID-19 pandemic. Turbine mechanical ventilators provide a powerful alternative to conventional ventilators, for example when there is a need to convert an existing hospital into a COVID-19 hospital or to setup COVID-19 intensive care wards within a hospital.

The turbine mechanical ventilator operates with an impeller to generate flow and pressure on demand. The impeller in operation draws ambient air from an inlet of a turbine in the ventilator to an outlet of the turbine. The ambient air may be mixed with oxygen or other gas that is selectively expelled into the turbine from a pressurized source. This mechanical design generates non interrupt flow of air from inlet to outlet optionally mixed with oxygen during operation of the impeller. Once the impeller operation is paused, some of the pressure generated in the turbine is released through the inlet creating a flow in an opposite direction, e.g., from the outlet to the inlet.

One of the operations periodically performed with a ventilator is called maneuvers or lung mechanics. During a maneuver, a defined pressure is applied to inflate a patient's air ways and the pressure is held over a period of a few seconds during which flow and pressure parameters are measured to determine a static compliance and a resistance of the patient's airways. One of the challenges of designing a turbine mechanical ventilator is how to enable holding the defined pressure while performing a maneuver with an impeller that cannot hold pressure. A known approach is to hold the pressure downstream from the turbine outlet when pausing operation of the impeller to perform the maneuver. For example, some turbine mechanical ventilators include a one way valve on the inspiratory port. This designs provides holding the pressure downstream the inspiratory port. Others, include an external one way valve mounted on an inspiratory port. It is also known to include a one way valve in the patient circuit, e.g. at the end of the inspiratory limb near the Y fitting. In each of these examples, the pressure is held downstream of the turbine.

### SUMMARY OF THE INVENTION

One of the disadvantages of the known turbine mechanical ventilators as compared to conventional ventilators is in the manner that maneuvers are performed. In known turbine mechanical ventilators, when the pressure is held with one way valves positioned downstream from the turbine, pressure and flow parameters are typically required to be sensed near the patient using pressure sensors located on the patient circuit. The pressure sensors added to the patient circuit are accompanied by additional cost and added complexity to the system.

Another disadvantage of the known turbine mechanical ventilators is that there is a tendency for some oxygen to be expelled into the ambient environment during operation of the ventilator. This may occur for example when the impeller operation is paused between breathing cycles and a mix of air and oxygen is released through the inlet. Leakage of oxygen is wasteful and potentially dangerous. For example, over time an enclosed room including one or more turbine mechanical ventilators may accumulate oxygen in dangerous levels.

According to an aspect of some example embodiments, the valve cassette and method for holding airway pressure in a turbine mechanical ventilator as described herein provides performing maneuvers while measuring flow and pressure parameters distally with respect to the patient and with sensors already available within the ventilator. This is similar to how maneuvers are performed with a conventional ventilator (driven with compressed air).

According to an aspect of some example embodiments, the valve cassette is configured for being mounted upstream an inlet to the turbine as opposed to downstream of a turbine outlet and a pressure is maintained within a housing around the turbine as well as downstream in the patient circuit for a defined period after pausing operation of the impeller. Since the pressure is also held upstream from the sampling ports within the ventilator, the sensors already included within the ventilator may be used for performing the maneuvers. The present inventors have found that use of the valve cassette as described herein may enable performing maneuvers with reduced cost and complexity of the system. Furthermore, the present inventors have found that the ability to block backflow through the valve cassette may provide additional advantages. For example, the pressure buildup due to the valve cassette may block oxygen from leaking out into the ambient environment over periods that the impeller operation is paused. Another advantage is that the buildup of pressure that is maintained by blocking backflow through the valve cassette is useful in preventing exhaled air from flowing into the patient circuit inlet and back to the patient over a subsequent inspiratory cycle.

According to an aspect of some example embodiments there is provided a valve cassette for a turbine mechanical ventilator comprising: a plurality of diaphragm check valves, wherein each of the plurality of diaphragm check valves includes a flexible membrane and a plug; a plate including a plurality of seats configured for receiving the plurality of diaphragm check valves, wherein each of the plurality of seats includes a bore for receiving the plug and a plurality of openings through which air is configured to flow through the valve cassette; and at least one connecting element configured for fixing valve cassette to a turbine housing inlet.

Optionally, the plurality of openings include a plurality of slits extending radially from a ring defining the central bore.

Optionally, an area of the plurality of openings is larger than area of an inlet to a turbine of the turbine mechanical ventilator.

Optionally, each of the plurality of seats includes an annular seat rim configured for engaging a perimeter of the flexible membrane.

Optionally, the plurality of diaphragm check valves are configured to seal the plurality of openings in a closed state and to allow air to be drawn through the plurality of openings into the turbine housing inlet in an open state.

Optionally, an outer facing surface of the plate is formed with ribs or protrusions, the outer facing surface being opposite an inner facing surface facing the turbine housing inlet.

Optionally, each of the plurality of seats is recessed with respect to an outer facing surface of the plate, the outer facing surface being opposite an inner facing surface facing the turbine housing inlet.

Optionally, the plurality of seats is arranged annularly on the plate.

Optionally, the at least one connecting element is a plurality of ear shaped elements extending from the plate, wherein each ear shaped element includes a screw hole for receiving a screw.

Optionally, the plate and the at least one connecting element are integral and formed with a polymer material in a blow molding process.

According to an aspect of some example embodiments, there is provided a turbine baffle housing for a turbine mechanical ventilator comprising: a first inlet port through which air is suctioned into the turbine baffle housing; a valve cassette installed over the first inlet with a sealing element configured for forming a sealed engagement, wherein the valve cassette as described herein; a second inlet configured for receiving compressed gas; a baffle arrangement configured for mixing flow from the first inlet and the second inlet and an outlet through which pressurized air is expelled.

According to an aspect of some example embodiments, there is provided method for performing a maneuver with a turbine mechanical ventilator, the method comprising: operating a turbine of the turbine mechanical ventilator to draw air through an air inlet of a housing including the turbine and build a defined pressure in the housing; blocking a backflow of air through the air inlet with a valve mounted on the air inlet; blocking release of air to the atmosphere through an exhaust of the turbine mechanical ventilator based on closing an exhalation valve controlling flow through the exhaust; pausing operation of the turbine based on reaching the defined pressure; sampling flow and sensing flow and pressure parameters from within the turbine mechanical ventilator while turbine operation is paused; and determining compliance of a patient's lung based on the sensing within the turbine mechanical ventilator.

Optionally, the valve mounted on the air inlet is a valve cassette including a plurality of one-way valves.

Optionally, the valve cassette as described herein.

Optionally, closing the exhalation valve is controlled based on pressurized flow sampled from within the turbine mechanical ventilator.

Optionally, the pressurized flow is configured to displace a diaphragm of the exhalation valve.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an exploded view of an example turbine mechanical ventilator in accordance with some example embodiments;

FIG. 2 is an exploded view of an example baffle housing for a turbine of the turbine mechanical ventilator in accordance with some example embodiments;

FIG. 3 is a perspective view of the example valve cassette installed on the example baffle housing in accordance with some example embodiments;

FIG. 4 is a perspective view of an inner volume of the example baffle housing and example flow during operation of the turbine in accordance with some example embodiments;

FIG. 5 is a perspective view of an inner volume of the example baffle housing and example accumulated pressure while the turbine is not in operation in accordance with some example embodiments;

FIGS. 6A and 6B are exploded views of the example valve cassette shown from the front and back respectively, both in accordance with some example embodiments;

FIG. 6C is perspective view of the example valve cassette shown from the front facing surface in accordance with some example embodiments;

FIGS. 7A and 7B are front and back view of the example valve cassette respectively in accordance with some example embodiments;

FIG. 8 is a simplified schematic diagram of flow through an example turbine mechanical ventilator and patient circuit in accordance with some example embodiments; and

FIG. 9 is a simplified flowchart of an example method to operating a turbine mechanical ventilator during maneuvers in accordance with some example embodiments.

### DESCRIPTION OF SPECIFIC EMBODIMENTS OF THE INVENTION

The present invention, in some embodiments thereof, relates to a turbine mechanical ventilator and, more particularly, but not exclusively, to a valve cassette for a turbine mechanical ventilator and method for holding airway pressure therewith.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

According to some example embodiments there is provided a valve cassette including a valve plate and a plurality of one way valves mounted thereon. According to some example embodiments, the valve cassette is configured to block backflow from the turbine through the inlet of a baffle housing including the turbine. According to some example embodiments, the valve cassette includes at least one connecting element configured for fixing the valve cassette to an inlet of the baffle housing. The valve cassette may be fixed over the inlet with a sealed connection so that air flow through the valve cassette only occurs through the one way valves. According to some example embodiments, the baffle housing is configured to direct ambient air toward the turbine optionally mixing the ambient air with pressurized gas, e.g. oxygen as it is being directed toward the turbine and then to direct air out of the baffling house through an outlet port fluidly connected to an inspiratory port on the ventilator.

According to an aspect of some example embodiments the baffle housing for a includes a first inlet port through which air is suctioned into the baffle housing, the valve cassette installed over the first inlet with a sealing element configured for forming a sealed engagement with the valve cassette, a second inlet configured for receiving compressed gas, a baffle arrangement configured for mixing flow from the first inlet and the second inlet and an outlet fluidly connected to the inspiratory port on the ventilator.

In some example embodiments, the one way valves on the valve cassette are diaphragm check valves. Optionally, the diaphragm check valves includes a flexible membrane and a plug. Optionally and preferably, the diaphragm check valves are formed with an elastomeric material, e.g. silicon, an elastomeric polymer, and/or rubber.

In some example embodiments, the valve cassette plate is formed with a plurality of seats, each configured for receiving a diaphragm check valve. In some example embodiments, the diaphragm check valve is fixed onto the valve cassette plate based on inserting the plug into the bore of the seat. According to some example embodiments, the seat additionally includes a plurality of openings through which air may be received in an open state of the valve. Optionally, the plurality of openings include a plurality of slits extending radially from a ring defining the bore. In some example embodiments, the seat includes an annular seat rim that engages a perimeter of the flexible membrane. Optionally and preferably, the at least one connecting element is integral to the valve cassette plate. Optionally and preferably, the valve cassette plate is formed with a polymer material, e.g. in a blow molding process or in an additive manufacturing process.

Optionally, the diaphragm check valves and the seats are arranged annularly on the valve cassette plate. By using a plurality of diaphragm check valves on the valve cassette plate as opposed to a single diaphragm check valves, the flow resistance through the valve cassette may be reduced and the response time of the valve cassette may be improved without compromising the ability of the valve cassette to prevent backflow. Optionally, the valve cassette includes 3-15 diaphragm check valves e.g., 4-6 diaphragm check valves and/or 5 diaphragm check valves.

According to some example embodiments, the plurality of seats are recessed with respect to an outer facing surface of the valve cassette plate. The outer facing surface is the surface that faces away from the turbine inlet. Optionally, the outer facing surface of the valve cassette plate is formed with a plurality of ribs or other protrusions. According to some example embodiments, the plurality of diaphragm check valve are mounted on an inner facing surface opposite the outer facing surface.

According to an aspect of some example embodiments there is provided a method for operating a turbine mechanical ventilator during maneuvers. The method includes connecting a patient to the ventilator, building a defined pressure with the baffle housing with the turbine, and stalling operation of the impeller of the turbine while maintaining the pressure buildup in the baffle housing and sensing pressure with pressure sensors housed in the ventilator housing. According to some example embodiments, the compliance and/or resistance of a patient's airway may be determined based on output from the pressure sensors. Optionally, a processor of the ventilator is configured to sample output from the sensors and determine the compliance and/or resistance.

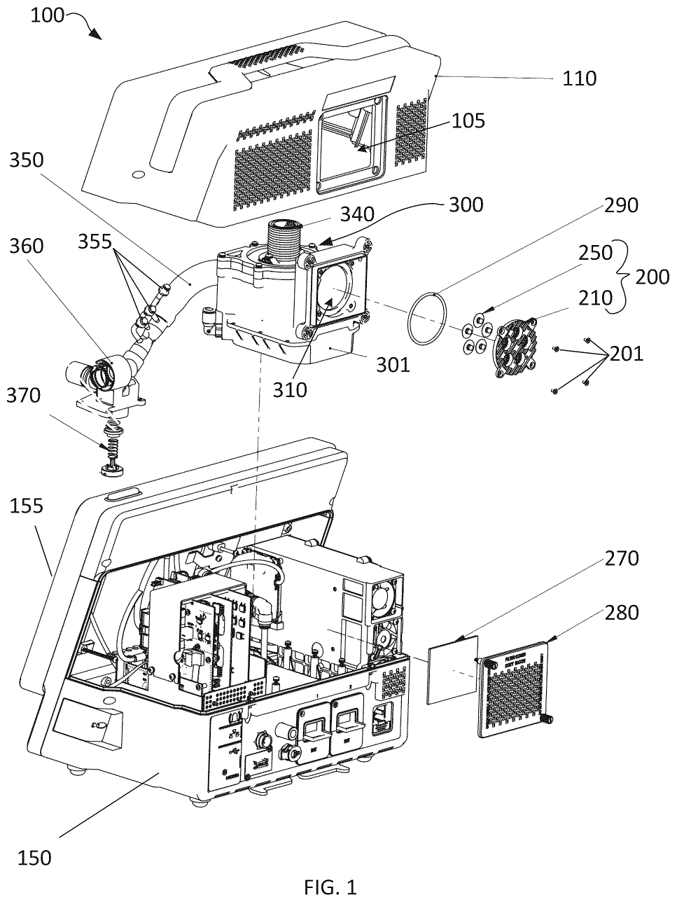

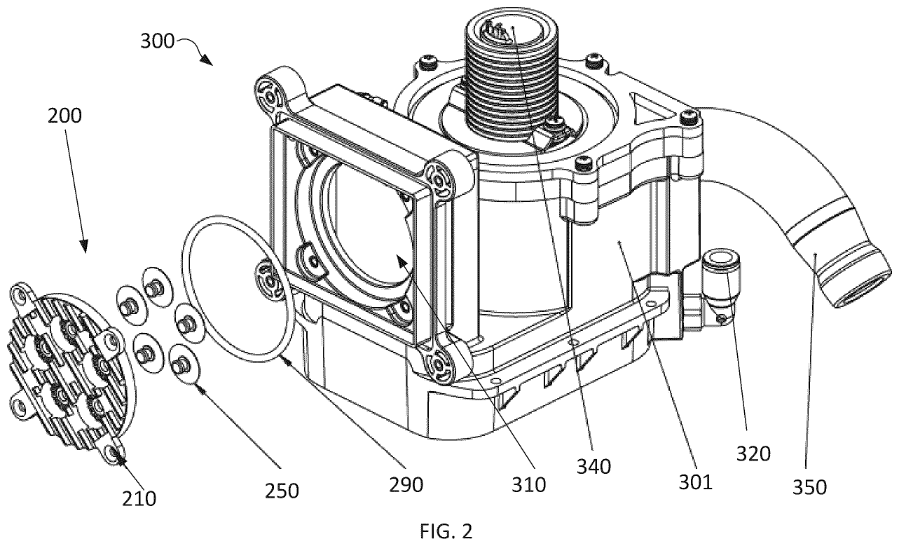

Reference is now made to FIG. 1 showing an exploded view of an example turbine mechanical ventilator and to FIG. 2 showing an exploded view of an example turbine of the turbine mechanical ventilator, both in accordance with some example embodiments. Turbine mechanical ventilator 100 includes a baffle housing 301 with turbine 300 installed in a ventilator housing 150 and covered with ventilator cover 110. A turbine motor 340 of turbine 300 may be installed on baffle housing 301 and extend outwardly. Air from the ambient environment may be drawn into baffle housing 301 through air inlet 310 typically aligned with opening 105 in ventilator cover 110. An air filter 270 and a filter cover 280 may cover opening 105 to filter air as it is being drawn into baffle housing through air inlet 310. Filter cover 280 may be fixed to ventilator cover 110, e.g. on a frame around opening 105.

In some example embodiments, baffle housing 301 additionally includes an inlet port 320 through which compressed gas, e.g. oxygen may be expelled into baffle housing 301 and optionally mixed with air drawn in through inlet 310. Outflow of pressurized air from baffle housing 301 may be through a tube 350 that extends from baffle housing 301 to an inspiratory port on a console 155 of ventilator 100. Tube 350 may be connected to the inspiratory port with base connector 360. Optionally, tube 250 is a flexible tube. Typically, tube 350 includes one or more sampling ports 355. Sampling ports 355 may connected to one or more sensors installed in ventilator housing 150 for monitoring pressure and flow during operation of ventilator 100. A pressure relief valve 370 may be installed on tube 350 as a safety measure to avoid generation of excess pressure that may potentially damage a patient's airways, e.g. lungs. In some example embodiments, pressure relief valve 370 is a mechanically operated valve, e.g. poppet valve.

According to some example embodiments, baffle housing 301 includes a valve cassette 200 installed over air inlet 310. According to some example embodiments, valve cassette 200 includes a plurality of diaphragm check valves 250 and a valve cassette plate 210 on which plurality of diaphragm check valves 250 are mounted. Diaphragm check valves 250 allow airflow into turbine 300 through inlet 310 and block backflow of air through inlet 310. According to some example embodiments, valve cassette 200 is secured over inlet 310, e.g. with one or more screws 201 and sealed against baffle housing 301 with a sealing element 290, e.g. an O-ring or gasket positioned between valve cassette 200 and a frame around air inlet 310. According to some example embodiments, valve cassette 200 provides holding a generated air pressure within baffle housing 301 while performing maneuvers to determine resistance and compliance of a patient's airways. Since valve cassette 200 blocks backflow at inlet 310, resistance and compliance of a patient's airways may be measured downstream from inlet 310 with existing sampling ports 355, sensors and a processor all housed in ventilator housing 150.

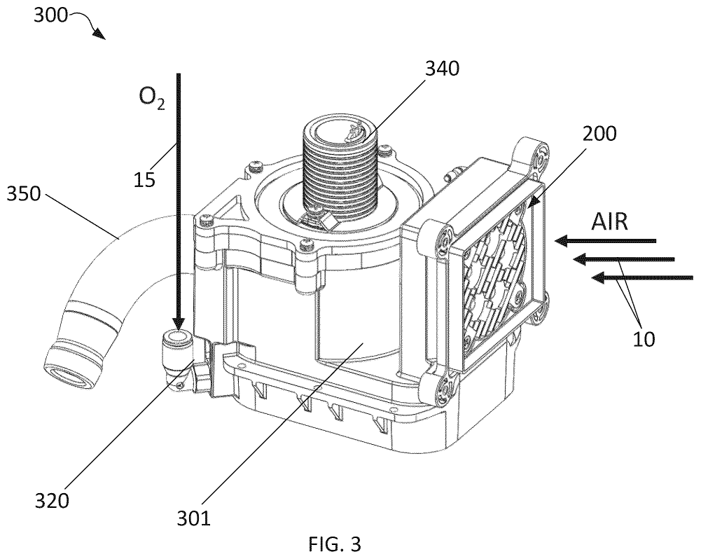

Reference is now made to FIG. 3 showing a perspective view of the example valve cassette installed on the example baffle housing in accordance with some example embodiments. During operation of turbine 300, air is drawn through valve cassette 200 into baffle housing 301. Concurrently, a compressed gas source, e.g. compressed oxygen may be connected to inlet port 320 and compressed gas may also flow into baffle housing 301. According to some example embodiments, the air and compressed gas is mixed in baffle housing 301 prior to being delivered to a patient through outlet tube 350. According to some example embodiments, air 10 penetrates into turbine through a plurality of one way valves mounted on valve cassette 200. Typically it is desired to provide a low resistance to flow through inlet for the purpose of reducing the energy required to draw in the required air as well as generate a desired pressure with a high response time. By using a plurality of one way valves 250 (FIG. 2) as opposed to a single one way valve, the resistance to flow into the turbine may be reduced, the response time and seal against backflow. According to some example embodiments, the total valve area provided by the plurality of one way valves 250 is selected to be greater than an inlet area in baffle housing 301 that directs air into turbine 300 so that valve cassette 200 does not increase resistance or does not substantially increase resistance of flow into turbine 300.

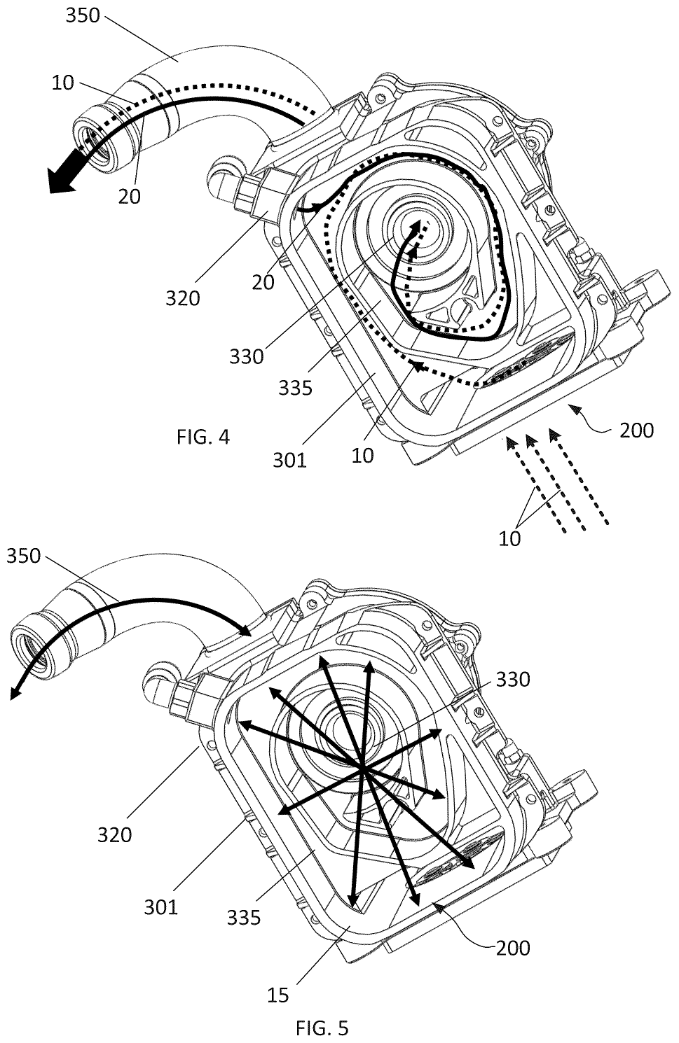

Reference is now made to FIG. 4 showing a perspective view of an inner volume of the example baffle housing and example flow during operation of the turbine in accordance with some example embodiments. Baffle housing 301 may include a series of baffles 335 configured for directing flow and mixing flow from inlet 310 and inlet 320 toward impeller 330 and through outlet tube 350. Air 10 drawn into baffle housing 301 through valve cassette 200 is ambient air, optionally and preferably filtered with air filter 270 (FIG. 1). According to some example embodiments, operation of impellor 330 provides suctioning air through valve cassette 200.

Reference is now made to FIG. 5 showing a perspective view of an inner volume of the example baffle housing and example accumulated pressure while the turbine is not in operation in accordance with some example embodiments. According to some example embodiments, valve cassette 200 provides holding air pressure 15 within baffle housing 301 and tube 350 while impeller 330 is turned off by blocking backflow through valve cassette 200. Based on this assembly, maneuvers may be performed using sensors embedded in ventilator housing 150 (FIG. 1) and with no additional need for external sensors and sensing ports.

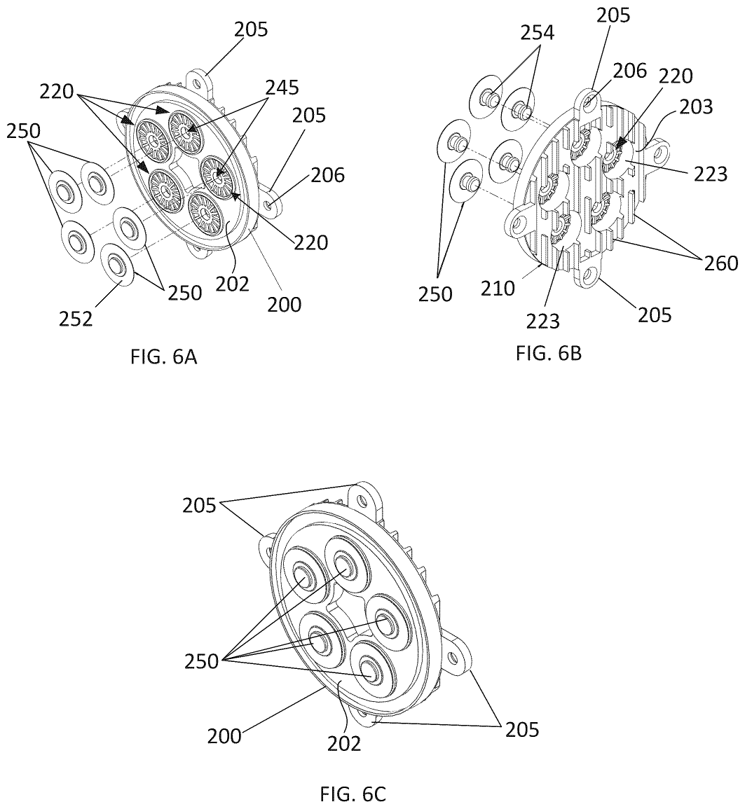

Reference is now made to FIGS. 6A and 6B showing exploded views of the example valve cassette shown from the front and back respectively and to FIG. 6C showing a perspective view of the example valve cassette shown from the front facing surface, all in accordance with some example embodiments. According to some example embodiments, valve cassette 200 includes a valve cassette plate 210 that is optionally and preferably formed from a polymer material in a blow molding process and a plurality of diaphragm check valves 250 that are optionally and preferably formed from an elastomer material, e.g. silicon. Optionally, valve cassette plate 210 is formed in an additive manufacturing process, e.g. with three-dimensional printer. According to some example embodiments, valve cassette plate 210 includes dedicated seats 220 for receiving diaphragm check valves 250. According to some example embodiments, each seat 220 includes a central bore 220 for receiving a plug 254 of a diaphragm check valves 250 and pattern of openings through which air can flow therethrough. Optionally and preferably, seats 220 are arranged annularly on valve cassette plate 210. Alternately seats 220 may be arranged in a grid pattern or otherwise distributed over plate 210. Optionally, valve cassette plate 210 includes 3-15 seats 220 and/or 4-6 seats 220, e.g. 5 seats 220.

According to some example embodiments, diaphragm check valves 250 are fitted onto seats 220 on a front facing surface 202 of valve cassette plate 210. Front facing surface 202 is the surface of valve cassette plate 210 that is positioned against sealing member 290 and inlet 310 (FIG. 1). In this manner, a flexible membranes 252 of diaphragm check valves 250 that cover seats 220 in a normally closed state can bend inwardly in an open state to expose a pattern of openings formed in seats 220 and allow air to penetrate therethrough.

In some example embodiments, seats 220 are recessed with respect to outer facing surface 203. Outer facing surface 203 is opposite inner facing surface 202 and typically faces filter 270 (FIG. 1). The recessed positioning of seats 220 forms a surrounding wall 223 around each seat 220 defining an air channel. In some example embodiments, outer facing surface 203 is formed with a plurality of ribs 260 or other protrusions. Ribs 260 may provide for distancing filter 270 from valve cassette plate 210 and thereby reduce resistance of flow toward seats 220.

In some example embodiments, valve cassette 200 includes a plurality of connecting elements 205 that extend out from valve cassette plate 210 and that are optionally and preferably in the form of ear shaped elements including screw holes 206 for fixing valve cassette 200 to baffle housing 301. In some example embodiments, connecting elements 205 are integral to valve cassette plate 210 and are formed as one piece in for example a blow molding process.

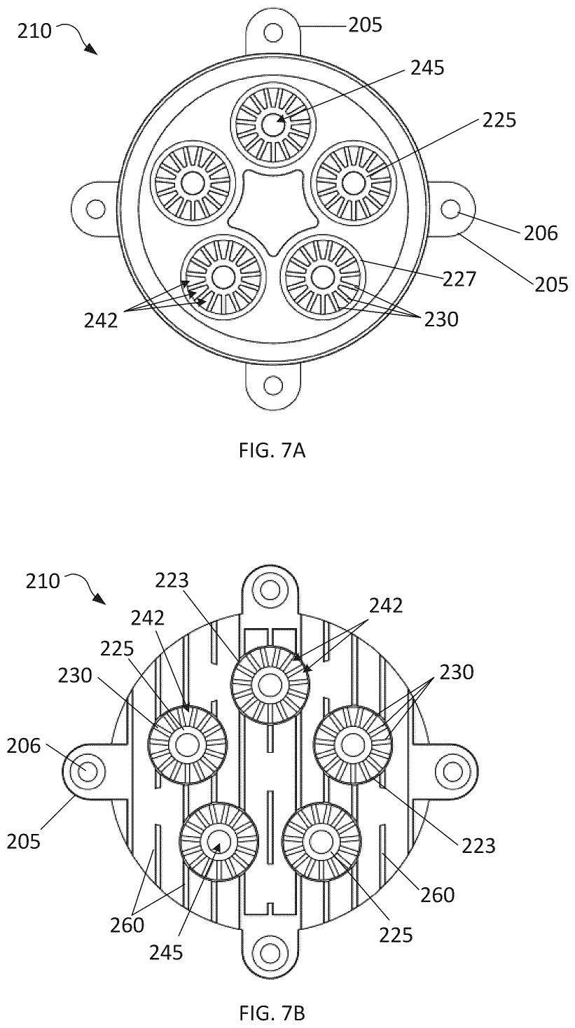

Reference is now made to FIGS. 7A and 7B showing front and back views of the example valve cassette respectively in accordance with some example embodiments. According to some example embodiments, each seat 220 includes a central bore 245 configured for receiving valve plug 254 (FIG. 6B), a plurality of openings 242 through which air flow may be received. Optionally, seats 220 additionally include a protruding rim 227 that defines the extent of seat 220 and physically engages a perimeter of flexible membrane 252 (FIG. 6A). In some example embodiments, plurality of openings 242 are formed between radial extensions 230 extending from a ring 225 defining bore 242 and rim 227. Radial extensions 230 may form an array of spokes. In other example embodiments, plurality of openings 242 may be formed by a different pattern, e.g. a grid shaped pattern. According to some example embodiments, an area of the plurality of openings 242 is defined to be larger than an inlet area into turbine 300.

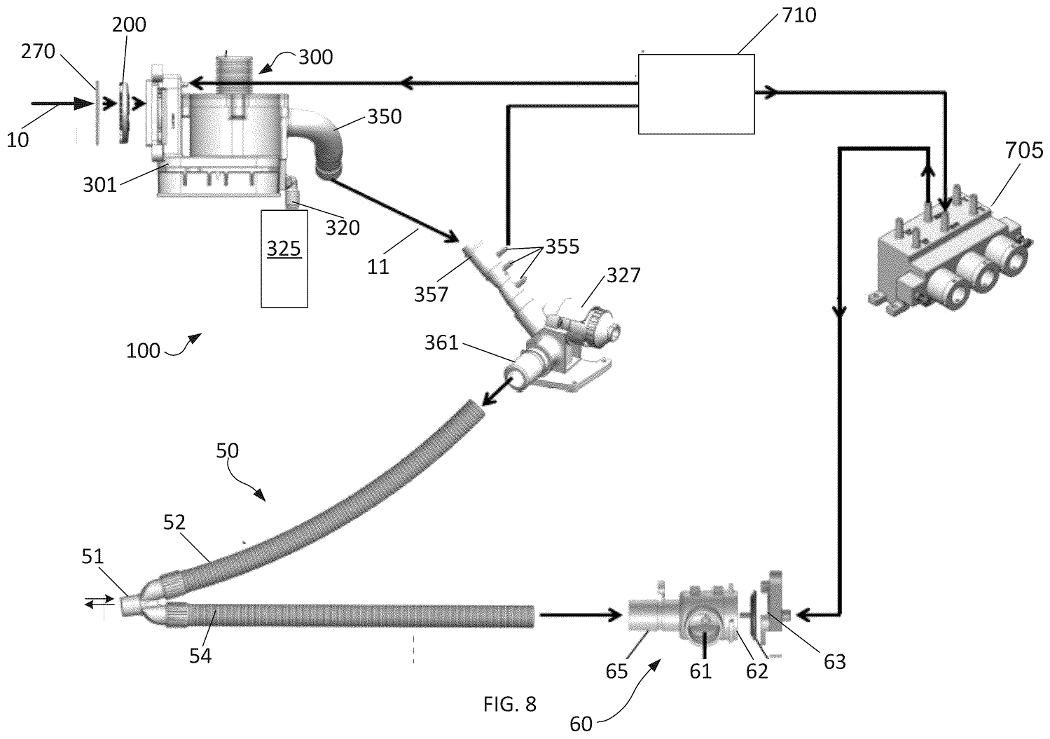

Reference is now made to FIG. 8 showing a simplified schematic diagram of flow through an example turbine mechanical ventilator and patient circuit in accordance with some example embodiments. According to some example embodiments, turbine 300 draws ambient air 10 into baffle housing 301 via a filter 270 and valve cassette 200. Optionally and preferably, a pressurized gas source 325, e.g. oxygen source concurrently releases gas into baffle housing 301 and the air is mixed with the released gas. The pressure buildup generates a flow of mixed air 11 through tube 350. Flow through tube 350 is directed through a sampling tube 357 including a plurality of sampling ports 355 for sampling flow within ventilator 100 and then released through an outlet port 361 on ventilator 100. Optionally and preferably, an oxygen level in mixed air 11 is also monitored with an oxygen sensor 327 fluidly connected to sampling tube 357. The air released through outlet port 361 is configured to be received by a patient via an inspiratory limb 52 of a patient circuit 50 connected to outlet port 361.

Typically, during an inspiratory cycle as well as during a maneuver it is desired to block air from flowing through exhalation limb 54 and escaping through exhaust 61. According to some example embodiments, the pressure build up in the system is used to actuate closing of exhalation valve 60 to prevent loss of pressure through exhalation limb 54. In some example embodiments, flow sampled from sampling ports 355 is also used to operate an internal exhalation valve 60. In some example embodiments, valves 710 selectively direct pressurized flow sampled from sampling ports 355 through an orifice 63 of exhalation valve 60. In some example embodiments the pressurized flow builds a back pressure that is configured to displace diaphragm 62 of valve 60 to a closed positioned. In this manner the generated air flow during an inspiration cycle and/or during a maneuver procedure is not lost through the exhalation limb 54. At the end of an inspiratory cycle and/or at the end of a maneuver procedure, valves 710 may be selectively controlled to block flow toward orifice 63 so that diaphragm 62 may revert to its normally open position. In an open position, air may be released through exhaust 61. In some example embodiments, ventilator 100 includes a valve housing block 705 that provides selectively toggling between internal controlling and/or sampling and external controlling and/or sampling.

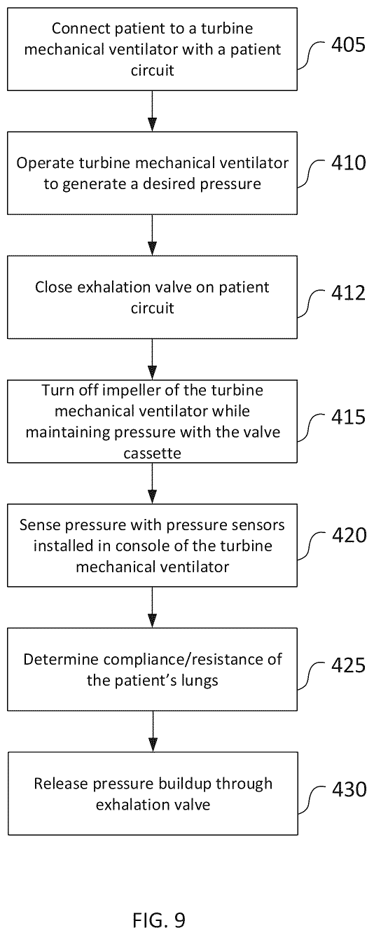

Reference is now made to FIG. 9 showing a simplified flowchart of an example method for performing maneuvers with a turbine mechanical ventilator in accordance with some example embodiments. During maneuvers a patient is connected to a patient circuit fitted on a turbine mechanical ventilator (block 405) and the turbine mechanical ventilator is operated to generate a desired pressure for inflating the patient's lungs (block 410). According to some example embodiments, the pressure build up additionally provides a control signal to an exhalation valve to close (block 412) and thereby block a release in the generated pressure through the exhalation limb of the patient circuit. When the desired pressure is achieved, the impeller of the turbine is turned off and the pressure generated is substantially maintained based on the valve cassette being installed on an air inlet of the turbine and the exhalation valve being closed (block 415). According to some example embodiments, pressure is sensed through sampling ports 355 connected to one or more sensors installed in ventilator housing 150 (block 420). Compliance and/or resistance of the lungs is determined by a processor in the ventilator housing 150 and is based on output from the sensors housed therein (block 425). At the termination of the maneuver procedure, the exhalation valve is opened and the pressure in the patient's lungs may be released (block 430).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. A valve cassette for a turbine mechanical ventilator comprising:
a plurality of diaphragm check valves, wherein each of said plurality of diaphragm check valves includes a flexible membrane and a plug;
a plate including a plurality of seats configured for receiving said plurality of diaphragm check valves, wherein each of said plurality of seats includes a bore for receiving said plug and a plurality of openings through which air is configured to flow through the valve cassette; and
at least one connecting element configured for fixing valve cassette to a turbine housing inlet.

2. The valve cassette according to claim 1, wherein said plurality of openings include a plurality of slits extending radially from a ring defining said central bore.

3. The valve cassette according to claim 1, wherein an area of said plurality of openings is larger than area of an inlet to a turbine of the turbine mechanical ventilator.

4. The valve cassette according to claim 1, wherein each of said plurality of seats includes an annular seat rim configured for engaging a perimeter of said flexible membrane.

5. The valve cassette according to claim 1, wherein said plurality of diaphragm check valves are configured to seal said plurality of openings in a closed state and to allow air to be drawn through said plurality of openings into said turbine housing inlet in an open state.

6. The valve cassette according to claim 1, wherein an outer facing surface of said plate is formed with ribs or protrusions, said outer facing surface being opposite an inner facing surface facing said turbine housing inlet.

7. The valve cassette according to claim 1, wherein each of said plurality of seats is recessed with respect to an outer facing surface of said plate, said outer facing surface being opposite an inner facing surface facing said turbine housing inlet.

8. The valve cassette according to claim 1, wherein said plurality of seats is arranged annularly on said plate.

9. The valve cassette according to claim 1, wherein said at least one connecting element is a plurality of ear shaped elements extending from said plate, wherein each ear shaped element includes a screw hole for receiving a screw.

10. The valve cassette according to claim 1, wherein said plate and said at least one connecting element are integral and formed with a polymer material in a blow molding process.

11. A turbine baffle housing for a turbine mechanical ventilator comprising:
a first inlet port through which air is suctioned into the turbine baffle housing;
a valve cassette installed over said first inlet with a sealing element configured for forming a sealed engagement, wherein said valve cassette is according to claim 1;
a second inlet configured for receiving compressed gas;
a baffle arrangement configured for mixing flow from said first inlet and said second inlet; and
an outlet through which pressurized air is expelled.

12. A method for performing a maneuver with a turbine mechanical ventilator, the method comprising:
operating a turbine of the turbine mechanical ventilator to draw air through an air inlet of a housing including said turbine and build a defined pressure in said housing;
blocking a backflow of air through said air inlet with a valve mounted on said air inlet;
blocking release of air to the atmosphere through an exhaust of the turbine mechanical ventilator based on closing an exhalation valve controlling flow through said exhaust;
pausing operation of said turbine based on reaching said defined pressure;
sampling flow and sensing flow and pressure parameters from within the turbine mechanical ventilator while turbine operation is paused; and
determining compliance of a patient's lung based on said sensing within the turbine mechanical ventilator.

13. The method of claim 12, wherein said valve mounted on said air inlet is a valve cassette including a plurality of one-way valves.

14. The method of claim 13, wherein said valve cassette comprises:
a plurality of diaphragm check valves, wherein each of said plurality of diaphragm check valves includes a flexible membrane and a plug;
a plate including a plurality of seats configured for receiving said plurality of diaphragm check valves, wherein each of said plurality of seats includes a bore for receiving said plug and a plurality of openings through which air is configured to flow through the valve cassette; and
at least one connecting element configured for fixing valve cassette to a turbine housing inlet.

15. The method of claim 12, wherein closing said exhalation valve is controlled based on pressurized flow sampled from within the turbine mechanical ventilator.

16. The method of claim 15, said pressurized flow is configured to displace a diaphragm of said exhalation valve.
